SEQUENCE LISTING (9 sequences)

SEQ ID NO: 1 - DNA, 279 bp, Artificial Sequence
  Mutated (recombinant) HPV-16 E7
atgcatggagatacacctacattgcatgaatatatgttagatttgcaaccagagacaactcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 2 - protein, 92 aa, Artificial Sequence
  Mutated (recombinant) HPV-16 E7
MHGDTPTLHEYMLDLQPETTQLNDSSEEEDEIDGPAGQAEPDRAHYNIVTFCCKCDSTLRLCVQSTHVDIRTLEDLLMGTLGIVCPICSQKP

SEQ ID NO: 3 - DNA, 38 bp, Artificial Sequence
  Oligonucleotide
gagaaaagagaggctgaagctcatggagatacacctac

SEQ ID NO: 4 - DNA, 35 bp, Artificial Sequence
  Oligonucleotide
gcaaccagagacaactcaattaaatgacagctcag

SEQ ID NO: 5 - DNA, 42 bp, Artificial Sequence
  Oligonucleotide
gttctcagaaaccataatgaattcatgtttcaggacccacag

SEQ ID NO: 6 - DNA, 33 bp, Artificial Sequence
  Oligonucleotide
gagggatccaatcatgcatggagatacacctac

SEQ ID NO: 7 - DNA, 35 bp, Artificial Sequence
  Oligonucleotide
gagaggcttctagaagattatggtttctgagaaca

SEQ ID NO: 8 - DNA, 34 bp, Artificial Sequence
  Oligonucleotide
gagggatccaatcatggagactctttgccaacgt

SEQ ID NO: 9 - DNA, 30 bp, Artificial Sequence
  Oligonucleotide
atcgatttgtcatatagacataaatccagt

(19) United States
(12) Patent Application Publication
(10) Pub. No.: US 2006/0099219 A1
(43) Pub. Date: May 11, 2006

(54) Mutated hpv-16e7 polypeptide, pharmaceutical composition comprising it and its preparation process

(75) Inventors: Sophie Hallez, Orbais (BE); Arsene Burny, Gembloux (BE); Alain Jacquet, Brussels (BE); Alex Bollen, Itterbeek (BE)

Correspondence Address: MERCHANT & GOULD PC, P.O. BOX 2903, MINNEAPOLIS, MN, 55402-0903, US

(73) Assignee: Universite Libre De Bruxelles, Brussels (BE)

(21) Appl. No.: 10/512,190

(22) PCT Filed: Apr. 24, 2003
(86) PCT No.: PCT/BE03/00073

§ 371 Date: Oct. 19, 2005

Related U.S. Application Data
(60) Provisional application No. 60/410,461, filed on Sep. 13, 2002.

(30) Foreign Application Priority Data
Apr. 24, 2002 (FR) 0205173
Apr. 3, 2003 (FR) 0304170

(51) Int. Cl. A61K 39/12; C12Q 1/70; C07H 21/04; C12N 15/86; C07K 14/025

(52) U.S. Cl. 424/186.1; 435/5; 435/69.3; 435/320.1; 435/456; 435/325; 530/350; 536/23.72

(57) ABSTRACT

The present invention is related to a mutated E7 polypeptide obtained from the human Papillomavirus HPV-16 as well as the pharmaceutical composition comprising it and its preparation process.

### FIELD OF THE INVENTION

The present invention is related to a mutated (recombinant) E7 polypeptide obtained from the human Papillomavirus (HPV) 16, as well as the pharmaceutical composition comprising it and its preparation process.

### SUMMARY OF THE INVENTION

Some specific cancers are induced by human Papillomaviruses (HPVs), especially the strain HPV-16.

Therefore, an aim of the present invention is to propose new antigens obtained from said human Papillomavirus that induce, when administrated to a patient, a specific human humoral and cellular response against said infectious agents, especially the strain HPV-16.

Therefore, a first aspect of the present invention is related to a mutated (recombinant) HPV-16 E7 polypeptide, wherein the sequence starting from amino-acid 21 and ending by amino-acid 26 of the (native) wild-type HPV-16 E7 (complete) polypeptide sequence is deleted and having the SEQ.ID No1.

The reference in amino-acid sequence of the (native) wild-type HPV-16 E7 polypeptide is preferably a sequence described by Seedorf et al. (1985), also described in the Swiss Prott Database under the No. P03129 and in the patent application WO00/03732.

The inventors have discovered that said mutated (recombinant) HPV-16 polypeptide (hereafter called E7 Δ21-26) presents, when administrated to a mammal patient (including a human), an unexpectedly improved (higher capacity than the corresponding (native) wild-type HPV-16 E7 polypeptide) capacity to induce a specific immune response in said patient, while reducing or blocking the immuno-suppressive characteristics of the corresponding (native) wild-type HPV-16 E7 polypeptide.

Said immuno-suppressive characteristics seem to be induced by said specific portion of the polypeptide starting from amino-acid 21 until amino-acid 26.

The present invention also includes variant or analog of said polypeptide wherein one or more other additional amino acids are modified or deleted from the complete HPV-16 E7 polypeptide sequence (without altering the immune response obtained). A variant of said polypeptide includes also molecules that have a similar pharmacological property than the mutated (recombinant) polypeptide according to the invention, preferably through the same biological pathway and acting similarly upon the same active site (recognized by the same antibodies or the same cell receptors).

The mutated (recombinant) polypeptide according to the invention can be formulated as “native protein” or is part of a “fusion protein” and may advantageously include additional amino-acid sequences that contain secretory or leader sequences, prosequences, sequences which elute in purification, such as multiple histidinoresidues or an additional sequence for stability during recombinant production (tag His in the C-terminal sequence).

Said mutated (recombinant) polypeptide may comprise also marker sequences which facilitate purification of the fusion protein with a sequence as an hexa-histidine peptide as provided in the PQE vector (Invitrogen Inc.) and described by Gentz et al., Proceeding National Academic of Science of the USA, 1989, Vol. 86, pp. 821-824) or an HA tag or glutathione-S transferase.

Said polypeptide could also be combined with other known sequences which are characterized by a specific induced immune response in a patient. A preferred example of fusion protein is a fusion of the polypeptide according to the invention (HPV E7 Δ21-26) with a tat polypeptide obtained from the HIV tat protein or tat derived peptide (i.e. tat 49-57 described hereafter).

A further aspect of the present invention is related to the polynucleotide (preferably the sequence SEQ.ID No.1) encoding the polypeptide or fusion protein according to the invention as well as the variants or fragments of said recombinant polynucleotide or fusion protein

The polynucleotide according to the invention could be also combined to one or more regulatory sequences controlling the expression of the polynucleotide according to the invention in a cell.

The corresponding polynucleotide or an expression cassette comprising said polypeptide may also contain non-coding 5′ and 3′ sequences such as non-translated sequences, splicing and poly-adenylation signals and ribosome binding sites or a marker for the selection of recombinant cells.

Another aspect of the present invention is related to a vector comprising the polynucleotide, the polypeptide or fusion protein according to the invention. Said vector is preferably a plasmid (preferably the chimeric plasmid is the PCDNA.3-Ii-E7 Δ21-26 or the pPIC9K described hereafter suitable for the transfection of a yeast such as Pichia pastoris), a virus, a liposome or a cationic vesicle able to transduct or transfect a cell and to obtain the expression and translation of said polynucleotide by said cell. Said vector may also comprise an inducible promoter such as the promoter AOX1 induced by glycerol.

A further aspect of the present invention concerns the cell (prokaryote or eukaryote cell such as yeast, preferably Pichia pastoris) transfected by or comprising said vector.

Preferably, the cells of the strain SMD 1168 (his4, pep4) of Pichia pastoris are used (Ref. C 175-00, Invitrogen).

Advantageously, the cells of Pichia pastoris are used, respectively:
GS115 (his4) (Ref.C181-00-Invitrogen; ATCC N°20864);
KM71 (his4; aox1; ARG4; arg4) (C183-00-Invitrogen, ATCC n°210078).

An additional aspect of the present invention is related to an inhibitor directed against the polypeptide, the fusion protein, a fragment (epitope) thereof or a polynucleotide encoding said polypeptide or fusion protein according to the invention.

Preferably, said inhibitor is an antibody (monoclonal or polyclonal antibody) or an active hypervariable portion thereof (Fab′₂, Fab, . . . ).

The inhibitor could be also a specific receptor of a blood cell able to interact specifically with said polypeptide or its epitopes.

The inhibitor could be also an antisense RNA or a ribozyme directed against the polynucleotide encoding said polypeptide and able to block the expression of said polynucleotide.

The present invention is also related to the cell (hybridoma) expressing and producing said antibody or an active hypervariable portion thereof.

A further aspect of the present invention is related to a pharmaceutical composition (including a vaccine) comprising an adequate pharmaceutical carrier (or diluent) and at least one of the various elements according to the invention, especially the polypeptide, its variant(s), the encoding polynucleotide(s), the vector, the cell transformed by said vector and/or the inhibitor according to the invention.

Advantageously, said pharmaceutical composition comprises the two polypeptides or fusion proteins according to the invention which present unexpectedly a synergetic effect when there are administrated (preferably simultaneously).

Said pharmaceutical composition may comprise also a suitable adjuvant, antioxidant buffer, bacteriostatic and solution which become biotonic with the blood of the recipient and aqueous and non-aqueous sterile suspensions (which may include suspension agents). The adjuvant used in the pharmaceutical composition is advantageously used for modulating the immune response of a mammal (including a human) in order to improve the characteristic of the pharmaceutical composition according to the invention or to reduce its possible side effects. The preferred adjuvant used according to the invention is the QuilA™ (Spikoside, Isotec AB lulea).

The suitable pharmaceutical carrier or diluent is selected by the person skilled in the art according to the type of administration to the mammal (oral administration, intravenous administration, intradermal administration, intramuscular administration, peritoneal administration, etc.).

The pharmaceutical composition can be present in a formulation in a unidose or multidose container and may be stored in a freeze dry condition which requires only the addition of a sterile liquid carrier.

Such pharmaceutical carrier could be in solid liquid or gaseous form and the suitable dose of administration and the ratio between the pharmaceutical carrier/active compound, varies according to the number of administration dose(s), the mass of the mammal to be treated and the possible side effects of the compound according to the invention upon said mammal.

Preferably, the pharmaceutical composition according to the invention is a prophylactic composition, such as a vaccine.

The present invention is also related to an immunological/vaccine formulation which comprises the polynucleotide (nude DNA) according to the invention presented according to the techniques well-known by the person skilled in the art such as the one described by Wolff et al. (Science, Vol. 247, pp. 1465-1468 (1999)).

The pharmaceutical composition according to the invention is advantageously used for the treatment and/or the prevention of cancers, especially cancers induced by a human Papillomavirus, in particular the strain HPV-16.

Another aspect of the present invention is related to a method of treatment or prevention of cancers affecting or supposed to affect a mammal (including a human), said method comprising the step of administrating to said mammal a sufficient amount of the pharmaceutical composition according to the invention, in order to prevent or cure either the symptoms of cancer or stop the development of tumors, in particular tumors induced human Papillomavirus, especially induced by HPV-16.

A further aspect of the present invention is related to the use of the pharmaceutical composition according to the invention for the manufacture of a medicament in the treatment and/or the prevention of cancer affecting or supposed to affect a mammal (including a human).

Another aspect of the present invention is related to the preparation process of the mutated (recombinant) HPV-16 E7 polypeptide or fusion protein according to the invention in a yeast cell, which comprises the following steps:
transformation of a culture of yeast cell(s), preferably Pichia pastoris, by the vector according to the invention,
selection of the cell(s) transformed by said vector,
culture in an adequate medium of said transformed cell(s) possibly with the addition of an inducing compound such as glycerol improving the production rate and,
purification of the mutated (recombinant) polypeptide or fusion protein according to the invention from the supernatant of cell(s) culture medium.

### DEFINITIONS

<<Polypeptide>> refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, PROTEINS—STRUCTURE AND MOLECULAR PROPERTIES, 2^{nd} Ed., T. E. Creighton, W. H. Freeman and Comany, New York, 1993 and Wolt, F., Posttranslational Protein Modifications: Perspectives and Prospects, pp. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth. Enzymol. (1990) 182 : 626-646 and Rattan et al., “Protein Synthesis: Posttranslational Modifications and Aging”, Ann NY Acad Sci (1992) 663 : 48-62.

“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double- stranded RNA, and RNA that is a mixture of single- and double-stranded regions, hybrid molecules comprising DNA and RNA that may be single-stranded or, more typically, double-stranded or a mixture of single- and double-stranded regions. In addition, “Polynucleotide” refers to triple-stranded regions comprising RNA or DNA or both RNA and DNA. The term “Polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions (preferably conservative), additions and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide. For instance, they should have similar antigenic or immunogenic activities as the reference polypeptide. Antigenicity can be tested using standard immunoblot experiments, preferably using polyclonal sera against the reference polypeptide. The immunogenicity can be tested by measuring antibody responses (using polyclonal sera generated against the variant polypeptide) against purified reference polypeptide in a standard ELISA test. Preferably, a variant would retain all of the above biological activities.

Fragments of polypeptide are also included in the present invention. A fragment is a polypeptide having an amino acid sequence that is the same as a part, but not all, of the amino acid sequence of the aforementioned polypeptide. Fragment may be “free-standing” or comprised within a larger polypeptide carrier protein (such as BSA) of which they form a part or region, most preferably as a single continuous region. Representative examples of polypeptide fragments of the invention, include, for example, fragments from about amino acid number 1-20, 21-40, 41-60, 61-80, 81-100, and 101 to the end of the polypeptide. In this context “about” includes the particularly recited ranges larger or smaller by several, 5, 4, 3, 2 or 1 amino acid at either extreme or at both extremes.

Preferred fragments include, for example, truncated polypeptides having the amino acid sequence of the polypeptide, except for deletion of a continuous series of residues that includes the amino terminus, or a continuous series of residues that includes the carboxyl terminus and/or transmembrane region or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments.

### DETAILED DESCRIPTION OF THE INVENTION

Production, purification and characterisation of protein E7 Δ21-26 and corresponding wild-type protein E7.

### Example 1

### 1) Cloning Procedure

To clone the (native) wild-type protein E7 in pPIC9K plasmid (Invitrogen, USA), a vector suitable for expression in Pichia pastoris, the starting material was plasmid pNIV5101. This recombinant plasmid consisted of the pMALcRI vector carrying the wild-type E7 gene from HPV16 (a gift of A. Burny, IBMM). The E7 module was isolated from pNIV 5101 after a concommitant digestion of the DNA with enzymes NcoI and SalI. The fragment of interest was made blunt-ended then inserted into plasmid pPIC9K, linearized with SnabI restriction enzyme. In this construction, the E7 protein, when expressed in Pichia pastoris, carried an excendentary tyrosine residue at its NH₂ - terminus. This residue was removed by site-directed mutagenesis (transformer site-directed mutagenesis kit from Clontec, USA) of the corresponding E7 DNA, using a synthetic 38-mer oligonucleotide having the following sequence: 5′ GAGAAAAGAGAGGCTGAAGCTCATGGAGATACACCTAC 3′ (The underlined sequence corresponds to the 3′ end of the signal sequence of the MFα factor placed upstream of the E7 DNA). The resulting plasmid pNIV5102 thus codes for the authentic wild-type E7 protein and, when introduced into P. pastoris, leads to the expression and secretion of said authentic protein.

Cloning of E7 Δ21-26 DNA was performed as follows, starting from pNIV5102. The sequence encompassing the (native) wild-type E7 DNA, including the MFu signal sequence, was recovered by digestion with BamHI and EcoRI (blunt-ended) and introduced by ligation into the pBlue script vector (Stratagene, USA) already cut with the same enzymes. The resulting plasmid was called pNIV5103. Site-directed mutagenesis was performed on this last plasmid in order to create the Δ21-26 deletion and to introduce an EcoRI restriction site downstream to the STOP codon of the E7 sequence. Two oligonucleotides were used therefore:

5′ GCAACCAGAGACAACT CAATTAAATGACAGCTCAG 3′ (the underlined codons correspond respectively to amino acid residue 20 and 27 of the E7 protein) and

5′GTTCTCAGAAACCATAATGAATTCATGTTTCAGGACCCCACAG 3′ (the underlined sequence corresponds to the creation of the EcoRI restriction site downstream to the stop codon of the E7 sequence).

The resulting plasmid, pNIV5109, thus carries the MFα factor—E7 Δ21-26 DNA sequence. A final construction consisted in cloning this DNA module into the expression vector for P. pastoris, pPIC9K, by ligation of corresponding DNA fragments cut with BamHI and EcoRI. The resulting expression plasmid was called pNIV5114.

### 2) Transformation of P. pastoris Yeasts and Expression Assay

Note: Procedures and precise composition of media used in these experiments are described in details in the Invitrogen (USA) manual (Multi copy Pichia Expression kit). The recipient P. pastoris strain SMD1168 (Invitrogen, USA) was transformed with the expression plasmid pNIV5114, linearized with the restriction enzyme BglII. The so-called spheroplast method was used. In short, yeasts were successively washed with water, SED medium and 1 M sorbitol. After centrifugation, washed yeasts were resuspended in SCE buffer and digested with zymolase according to the protocol given in Invitrogen's instruction manual. Resulting spheroplasts were recovered by centrifugation (750 g, 10 min), washed with 1 M sorbitol and then with Ca S buffer, before being used for transformation (10 μg of pNIV5114 DNA cut with BglII). Transformed yeasts were spread onto RBD gelose medium on top of which a layer of soft agar (so-called RD soft agar in the manufacturer's manual).

His⁺ Transformants (able to grow in absence of histidine in the medium) were then picked and spread onto YPD agar plates containing increasing concentrations of the antibiotic G418, (from 0,5 to 4 mg/ml) to select yeast transformants having incorporated several copies of the MFα—E7 Δ21-26 DNA sequence.

G418—resistant colonies were then tested for expression and secretion of the product of interest. Yeast colonies were grown in BMGY medium (Invitrogen's manual) up to an OD_{600 nm} comprised between 2 and 6. At that time, part of the cultures was centrifuged; the cell pellet was resuspended in BMGY medium supplemented with methanol (final concentration 0.5%) to induce expression and then grown again for up to several days. Aliquots were taken at different times post-induction and assayed for expression and secretion. The presence of protein E7 Δ21-26 in spent culture medium was detected by staining of proteins separated on polyacrylamide gels and by Western blotting using a monoclonal antibody against protein E7 (Santa Cruz, USA, dilution 1/1000). One of yeast transformants, pNIV5114 n°1, considered as the best secreting one, was chosen and used to construct a Master Cell Bank according to GLP procedures. From one vial of the MCB, a Working Cell Bank (WCB) was realized and served for the routine production of the E7 Δ21-26 protein.

### 3) Production of E7 Δ21-26 Protein in Bioreactors

A 100 ml preculture of recombinant yeast, pNIV5114 n°1 (150 μl of WCB vial), was prepared in BMGY medium and grown at 30° C. for 24 hours under shaking (250 rpm) up to an OD₆₀₀ of ±30. The preculture was used to inoculate a 2 L bioreactor (Bioflo III, New Brunswick, USA), containing 1.75 litres of Basal Salt Medium pH5, supplemented with antifoaming agent (0.5 ml) and PTM₁ salts (8 ml). Yeasts were allowed to grow at 30° C under agitation. The dissolved oxygen concentration was maintained at 20%, by controlling agitation speed (between 300 and 1000 rpm). When a first peak of oxygen consumption appeared (±18 hours post inoculum), 150 ml of glycerol 50% was added to the culture and the pH was adjusted to 6. About 3 hours later, when a second peak of oxygen consumption occurred, 10 g of methanol were added to the culture. The methanol concentration was then maintained at 5 g/liter for the whole induction period (25 to 30 hours). Thereafter, the biomass was removed by centrifugation (30 min at 1000 rpm) and the spent culture medium containing the product of interest was filtered through a Sartopure PP2 filter (1.2 μm porosity).

(Note: the fermentation of the (native) wild-type E7 protein followed the same procedure as described here for the mutant E7 Δ21-26 protein).

| Media | BMGY | |
|---|---|---|
| Yeast extract | 10 | g/L |
| Peptone | 20 | g/L |
| Potassium phosphate buffer | 100 | mM pH 6 |
| YNB with (NH₄)₂SO₄ but lacking amino acids | 13.4 | g/L |
| (Invitrogen, USA) | | |
| Biotine | 400 | μg/L |
| Glycerol | 10 | m/L |
| Basal Salts Medium | | |
| Phosphoric acid | 26.7 | ml/L |
| Calcium sulfate | 0.93 | g/L |
| Potassium sulfate | 18.2 | g/L |
| Magnesium sulfate (7H₂O) | 14.9 | g/L |
| Potassium hydroxide | 4.13 | g/L |
| Glycerol | 40.0 | g/L |
| PTM₁, Trace salts | | |
| Cupric sulfate [symbol] 5H₂O | 6.0 | g/L |
| Sodium iodide | 0.08 | g/L |
| Manganese sulfate [symbol] H₂O | 3.0 | g/L |
| Sodium molybdate [symbol] 2H₂O | 0.2 | g/L |
| Boric acid | 0.02 | g/L |
| Cobalt chloride | 0.5 | g/L |
| Zinc chloride | 20.0 | g/L |
| Ferrous sulfate [symbol] 7H₂O | 65.0 | g/L |
| Biotin | 0.2 | g/L |
| Sulphuric acid | 5.0 | ml/L |

### 4) Purification of E7 Δ21-26 Protein

Spent culture medium was diluted 4 fold with water and the pH was adjusted to 7.5. The fluid was immediately applied onto a Q sepharose XL column (2.6 cm×12 cm) equilibrated in 20 mM Tri-HCl pH 7.5 and flowing at 30 ml/min. The column was then washed successively with the equilibration buffer and with the same buffer containing 400 mM NaCl. Elution of the protein of interest was achieved by applying a NaCl gradient (400 to 650 mM), corresponding to 15 volumes of the column. Fractions containing the E7 proteins were pooled, concentrated by ultrafiltration (Amicon YM10 membrane) and applied onto a molecular sieving column Superdex 200 (1.6×60 cm) equilibrated in PBS buffer pH 7.2. Fractions corresponding to the E7 proteins were pooled and tested for endotoxin content. Whenever necessary, endotoxins were removed by treatment with Triton X-114. Protein concentration was measured by the Micro BCA assay (Pierce). The pure protein material was stored at −20° C. The analysis on polyacrylamide gel indicated that the E7 Δ21-26 protein consists of 2 immunoreactive bands having molecular masse of 14 and 16 Kda respectively. The stability of E7 Δ21-26 was measured in an accelerated stability assay. After 33 days at 30° C., the protein still presented the normal electrophoretic pattern and the normal immunoreactivity. Additional experiments later showed that E7 Δ21-26 was stable at −20° C. and at +4° C. for several months. At last, it was shown that the E7 Δ21-26 protein had an isoelectric point of 4.5.

(Note: the (native) wild-type E7 protein was purified essentially as described for the mutant E7 Δ21-26 protein but required an additional step to reach comparable purity).

### 5) Immunological Methods

### Mice and Cell Lines

6 weeks-old, female C57BL/6 (H-2^{b}) mice (Harlan, NL) were used in the experiments. C3 cells (Feltkamp et al, 1993), deriving from C57BL/6 embryonic cells and transformed with the full HPV16 genome together with the ras antigen, were cultivated in DMEM medium (Biowhittaker), supplemented with 10% Fetal Calf Serum, 50 μg/ml Penicillin, 50 μg/ml Streptomycin and 250 ng/ml Fungizone at 37° C., in humid atmosphere containing 7 % CO₂. For in vivo experiments, cells, collected by trypsin treatment from culture dishes, were washed in medium without serum before being injected into mice.

### 6) Measure of Anti Tumoral Effect

Groups of 8 mice were injected twice subcutaneously at the base of the tail with sample of HP16 E7 proteins produced in P. pastoris (i.e. 7.3 μg wild-type E7, 6, 9 μg mutant E7 Δ21-26) or in E. coli (ref: Hallez et al, 1999, 10 μg His₆-E7). Proteins were adjuvanted with Quil A (15 μg) (Brenntag Biosector, Denmark). The negative control injection consisted of Quil A in PBS buffer. Volumes injected were consistently of 100 μl. Two weeks after the second administration of the E7 proteins, mice were injected with 500.000 C3 cells, sub-cutaneously in the flank. The tumoral growth was evaluated once a week by measuring the diameters of the tumors and the average tumoral diameter was calculated.

### 7) Humoral Immunity

Seric antibodies against E7 were detected by ELISA. 96 wells plates (F96 Maxisorp, Nunc, Roskilde, Denmark) were coated overnight with the Hiss E7 protein (5 μg/ml in PBS buffer). After washing with PBS buffer, coating of the wells was blocked by 1% BSA (bovine serum albumin, Sigma) in PBS for 1 h at 37° C. Plates were then washed again with PBS before the addition of antisera, serially diluted 5 fold in 0.1% BSA/PBS, and incubated overnight at 4° C. Monoclonal antibodies, anti E7 HPV-TVG710Y (IgG2a) and ED17 (IgG1) were used as standards. After washing, specifically bound IgGs were detected with a peroxidase-labelled sheep antibody raised against murine IgGs (Amersham), used at the 1/2000 dilution in 0.1% BSA/PBS. Anti-isotypic antibodies, peroxidase-labelled (LO-IMEX, Belgium), were used to detect IgG1 and IgG2b anti E7 γ-globulins. After 1 hour of incubation at 37° C., plates were washed and the peroxidasic activity was detected and measured via the o-phenylene diamine substrate. The antibody titer is expressed as the inverse of the dilution giving an A₄₉₀ value of 0.6.

### 8) Measure of the Therapeutic Anti-Tumoral Response

Mice (n=24) were first injected (s.c. in the flank) with 500.000 C3 cells (day 0). On days 2 and 7, mice were injected, s.c. at the base of the tail, with Quil A-adjuvanted E7 proteins. The first group (n=8) received wild-type E7 (7.3 μg), the second, E7 Δ21-26 (6.9 μg) and the third one, only Quil A in PBS (100 μl) as control. The size of the tumors was measured twice a week as described above.

### 9) Cellular immunity and Cytokine Assays

Spleens of 3 mice from each group eight were collected two weeks after the second immunization with E7 proteins. Splenic cells were isolated and resuspended in DMEM medium supplemented with 1% normal mouse serum (Harlan), 50 IU/ml Penicillin, 50 μg/ml Streptomycin, non-essential amino acids, 2 mM L-glutamine, 10 mM Hepes and 5.10⁻⁵ M mercapto ethanol. Cells were dispersed into 96 flat-bottom wells (500.000/well) and stimulated or not with 9 μM of E7-derived peptides (E7₄₉₋₅₇, E7₄₁₋₆₂; Eurogentec, Belgium), 8 μg/ml E7 wild-type or 10 μg/ml His₆ E7 and SEB (30 ng/ml) (references: Feltkamp et al, 1993; Tindle et al, 1995). After 24, 48 and 72 hours of incubation at 37° C. in humid atmosphere with 7% CO₂, supernatant of wells was collected, frozen at −70° C. and later tested for the content in cytokines IL2, IL-4 and IFN-γ. IFN-γ was quantified using an ELISA assay based on monoclonal antibodies F1 and Db-1 (LO-IMEX, Belgium) as previously described (ref: De Smedt et al, 1996). IL-2 and IL-4 were quantified by ELISA using for capture and detection, respectively the pairs of monoclonal antibodies BVD4-1D11/BVD6-24G2 and JES6-1A12/JES6-5H4 (Pharmingen). Lymphoproliferation was measured after addition of 0.4 μCi 3H-thymidine in the wells and 16 hours of culture. Cells taken 24 and 48 hours later were lysed and the lysate was filtered on glass filter. Radioactivity incorporated into DNA was then measured in a liquid scintillation 0 counter.

### Results

### Advantages of E7 Δ21-26 in terms of Process Development and Industrial Utility

After several rounds of fermentation, and several purification runs, as described above, it appeared that:
in similar conditions, the productivity of the yeast strain secreting E7 Δ21-26, during the methanol-induction period, was significantly better (two fold) than that of the yeast strain secreting the wild-type E7 protein.
The purification protocol, when applied to E7 Δ21-26 was sufficient to yield a pure protein whereas the E7 wild-type still contained significant amounts of contaminants. To reach comparable purity with E7 (native) wild-type, a denaturing step (in guanidinium chloride) had to be performed after the Q sepharose chromatography and before the molecular sieving step.

### Injection of E7 (Native) Wild-Type and E7 Δ21-26, Adjuvanted With Quil A, Generate a Protective Anti-Tumoral Response in Mice (Preventive Vaccination)

Two injections of E7 wild-type/Quil A and of E7 Δ21-26/Quil A, three weeks apart, led to the protection against the HPV16 positive C3 syngenic tumor.

Three independent experiments have been performed. The results shown in table 1 indicate that both recombinant proteins, E7 (native) wild-type and E7 Δ21-26, induce resistance to progression of the syngenic tumor, i.e. generate a protective antitumoral immunity. The table 1 also shows that E7 Δ21-26 is a better immunogen than the wild-type (native) E7 polypeptide.

### Injection of E7 Wild-Type and E7 Δ21-26, Adjuvanted With Quil A, Generate a Therapeutic Antitumoral Response in Mice (Therapeutic Vaccination)

The rejection of pre-implanted tumors in mice was tested using Quil A-adjuvanted E7 (native) wild-type and E7 Δ21-26 proteins. As seen in Table 2, tumor regression or tumoral growth inhibition were observed with E7 wild-type protein whereas total tumor rejection or tumor growth inhibition occurred by vaccination with E7 Δ21-26. Clearly, E7 Δ21-26 protein led to a significantly longer survival in vaccinated animals than E7 (native) wild-type, indicating that E7 Δ21-26 is a better therapeutic immunogen than its wild-type counterpart.

### E7 Wild-Type and E7 Δ21-26 Proteins, Adjuvanted With Quil A, Generate Immune Responses In Vivo (Humoral and Cell-Mediated Immunity)

### a) Cell-Mediated Immunity

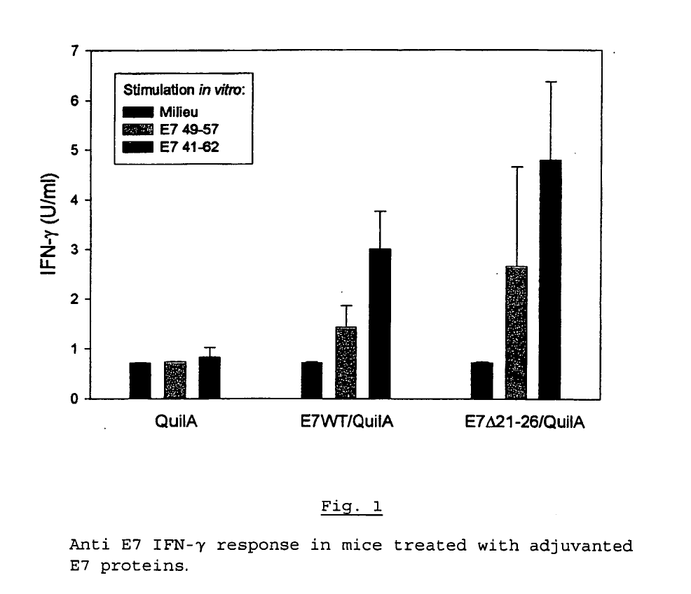

The stimulation of mouse splenic cells in vitro by E7 epitopes (MHCI by peptide E7₄₉₋₅₇ or MHCII by peptide E7₄₁₋₆₂) led to the synthesis of IFN-γ, but not of IL-2 or IL-4, by CD8⁺ and CD4⁺ anti E7 T lymphocytes. Splenocytes of immunized mice produced 2 to 7 fold more IFN-γ after stimulation with E7 peptides than without specific stimulation (fresh culture medium). FIG. 1 summarizes the results and clearly shows that the E7 Δ21-26 protein elicited stronger T CD8⁺ (2.75 fold) and T CD4⁺ (1.82 fold) responses than the wild-type protein, indicating therefore that E7 Δ21-26 is a better immunogen than its wild-type counterpart.

### b) Humoral Response

The presence of antibodies against the E7 protein was observed in the sera of animals vaccinated twice at 3 weeks intervals. For both E7 proteins tested, E7 wild-type and E7 Δ21-26, antibody titers in animals were variable from experiment to experiment and from animal to animal, indicating that there was no correlation between the titer of immunoglobulins and the resistance to tumor growth. In terms of isotype, the humoral response was characterized by IgG2b and IgG1 species, for both antigens.

### Conclusion

1) Administration of E7 wild-type and E7 Δ21-26 proteins to mice induced both humoral and cell-mediated immunity. The E7 Δ21-26 antigen induced a IFN γ, E7 specific response, higher than the wild-type counterpart, which contributes to its stronger anti-tumoral activity.

2) E7 Δ21-26 protein has moreover three additional advantages on the wild-type species.

a) expression level in Pichia pastoris higher than with wild-type

b) purification yield and easiness much better than with wild-type (less contaminants)

c) deletion Δ21-26 largerly attenuates the potential oncogenic nature of wild-type E7.

### Example 2

### 1) Production of E7(Δ21-26)-Tat Fusion Protein and Study of the Therapeutic Anti-Tumor Effects of E7(Δ21-26)-Tat/Adjuvant Injections

Improving cancer vaccination mostly relies on increasing the generation of tumor Ag-specific cytotoxic T-cells (CTL). Proteins are particularly safe to elicit immunity against oncoproteins but generate weak and predominantly humoral immune responses. Next to co-administration with adjuvant, protein modifications targeting them to Ag presenting cells, have allowed the induction of specific CTL and anti-tumor responses.

Because several studies reported that exogenous proteins, linked to full length HIV-1 Tat protein or Tat-derived peptides as short as Tat₄₉₋₅₇ (tatmin comprising only the sequence starting from amino acid 49 until amino acid 57), penetrate into cells, are presented onto MHC-I molecules and generate specific CTL responses in vitro, it might be useful to test this strategy with the E7 Δ21-26 antigen. The expectation is that fusing this antigen to Tat-transduction domain would enhance tumor-directed immune responses (references: Vocero-Akbani et al, 2000; Kim et al, 1997; Moy et al, 1996).

### 2) Genetic Constructs

The E7 Δ21-26 protein can be expressed in fusion with the ‘Tat minimal region’ and as a secreted form in P. pastoris. The DNA cassette encoding E7 (Δ21-26)-Tatₘᵢₙ can be inserted into the pPIC9K expression vector downstream to the S. cerevisae α mating factor signal sequence (MFα).

### 3) Protein Production in P. pastoris, Purification and Characterization

pPic9K-E7 (Δ21-26)-Tatₘᵢₙ can be introduced into P. pastoris using the spheroplast transformation method. After the screening of His⁺ transformants, clones carrying multiple copies of E7 Δ21-26-Tatₘᵢₙ coding cassette can be isolated after a geneticin (G418) resistance test. Resistant clones can be cultured and E7 (Δ21-26)-Tatₘᵢₙ can be purified by a combination of anion exchange and gel filtration chromatographies and subsequently characterized.

Test of E7 (Δ21-26)-Tat/adjuvant (Quil A)-induced systemic immune responses. Antibody response can be tested by measuring anti-E7 (E2) IgG, IgG2b, IgG2c and IgG1 titers in the serum (ELISA). Cell-mediated immunity can be measured using spleen, lymph node and peripheral blood cells in vitro sensitized with MHC-I and -II Ag-derived epitopes. Activation of specific T helper response can be tested by measuring lymphoproliferation and production of Th1-(IL-2, IFN-γ) and Th2-(IL-4, IL-10) type cytokines: ELISA, cytokine flow cytometry (CFC). CD8⁺ T cell response can be monitored by enumerating those producing IFN-γ and TNF-α (CFC) and by testing their lytic activity.

### 4) Test of Therapeutic Anti-Tumor Effects

The C3 cells (C57BL6 origin, H-2^{b}), which are embryonic cells transformed with HPV16 and ras, can be used as a tumor model (Feltkamp et al, 1993). The immunotherapeutic potential of the mutated Tat fusion proteins can be tested by injecting them mixed with Quil A to mice bearing pre-implanted C3 tumors and by recording the percentage of cured mice. Their respective curative potential can be compared to those elicited by E7 (Δ21-26)/QuilA and His₆-E7-Tat₄₉₋₈₆/QuilA vaccines.

### 5) Genetic Immunisation (NAVAC)

NAVAC, or genetic immunization, offers another possibility to generate efficient immune responses against tumor development. The strategy consists of targeting the E7 Δ21-26 DNA sequence towards the endosome where efficient processing into peptides will occur in the context of the MHC II pathway. The targeting uses a driver which is the invariant chain DNA sequence that can, after fusion to the sequence encoding E7 Δ21-26 and with the help of a ‘naked DNA’ expression plasmid, increase the cellular immunity of the host against tumor.

A chimeric plasmid is constructed: it consists of:

a) the ‘expression’ plasmid suitable for genetic immunization, pCDNA3. zeo (Invitrogen)

b) the DNA sequence encoding for the amino acids 1 to 80 from the Invariant chain, ligated downstream to the promoter carried by the expression vector.

c) The DNA sequence for E7 Δ21-26 fused in frame and downstream to the sequence of the Invariant chain.

The chimeric plasmid pCDNA.3-Ii-E7 Δ21-26 can be propagated and prepared according to State of the Art techniques.

TABLE 1
Preventive vaccination of mice with E7 Δ 21-26
and E7 wild-type proteins

| | | | Survival | |
|---|---|---|---|---|
| Group | Adjuvant | Antigen | (3 independent experiments) | % |
| I | Quil A | E7 wild-type | 16/24 | 66.6 |
| II | Quil A | E7 Δ 21-26 | 22/24 | 91.6 |
| III | Quil A | None | 0/24 | 0 |

TABLE 2
Therapeutic vaccination of mice with E7 Δ 21-26 and E7 wild-type proteins

| | | | | | | Mean survival time |
|---|---|---|---|---|---|---|
| | | | Complete | Transitory | Inhibition of | (days) |
| | | | tumor | tumor | tumor growth | (Average tumor |
| Group | Adjuvant | Antigen | rejection | regression | rate | diameter = 20 mm) |
| I | Quil A | E7 wild-type | 0 | 2/8 | 6/8 | 28 ± 6 (P = 0.0016) |
| II | Quil A | E7 Δ 21-26 | 1/8 | 7/8 | 0 | 42 ± 8 |
| III | Quil A | none | 0 | 0 | 0 | 26 ± 3 (P = 0.0001) |

### References Cited in the Text

1) De Smedt, T., et al., J. Exp. Med. 184:1413-1424 (1996).
2) Feltkamp, M. C., et al., Eur. J. Immunol. 23:2242-2249 (1993).
3) Hallez, S., et al., Int. J. Cancer 81:428-437 (1999).
4) Tindle, R. W., et al., Clin. Exp. Immunol. 101:265-271 (1995).
5) Kim, D. T., et al., J. Immunol. 159, 1666-1668 (1997).
6) Feltkamp, M. C., et al., Eur. J. Immunol. 23:2242-2249 (1993).
7) Moy, P., et al., Mol. Biotechnol. 6,105-133 (1996).
8) Vocero-Akbani, A., et al. Methods Enzymol. 2000. 322:508-521.

1. A mutated HPV-16 E7 polypeptide wherein the sequence starting from amino acid 21 and ending by amino acid 26 of the native wild-type HPV- 16 E7 complete polypeptide sequence is deleted.

2. The mutated polypeptide of claim 1, having the sequence SEQ.ID No.1.

3. A fusion protein comprising the mutated HPV-16 E7 polypeptide sequence according claim 1, linked to a tat peptide, preferably the HIV-1 tat peptide.

4. The fusion protein of claim 3 wherein said tat peptide is a tat (49-57) peptide.

5. A polynucleotide encoding the recombinant polypeptide according to claim 1 a fusion protein comprising the mutated HPV-16 E7 polypeptide sequence according to claim 1, linked to a tat peptide, preferably the HIV-1 tat peptide.

6. The polynucleotide according to claim 5, which further comprises one or more regulatory sequences controlling the expression of the polynucleotide into a cell, preferably a yeast cell such as Pichia pastoris.

7. A vector comprising the polynucleotide, the polypeptide or the fusion protein according to claim 1.

8. A cell transfected by or comprising the vector according to the claim 7.

9. The cell according to the claim 8, being a yeast, preferably Pichia pastoris.

10. An inhibitor directed against the polypeptide, the fusion protein or the polynucleotide according to claim 1.

11. The inhibitor according to the claim 10, being an antibody (monoclonal or polyclonal antibody) or an active hypervariable portion thereof.

12. An inhibitor directed against the polypeptide. the fusion protein or the polvnucleotide according to claim 1, being an antisense RNA or a ribozyme directed against the polynucleotide encoding the recombinant polypeptide according to claim 1 or the fusion protein according to a fusion protein comprising the mutated HPV-16 E7 polypeptide sequence according to claim 1, linked to a tat peptide preferably the HIV-1 tat peptide.

13. A hybridoma cell expressing and producing the antibody according to the claim 11 or an active hypervariable portion thereof.

14. A pharmaceutical composition comprising an adequate pharmaceutical carrier (or diluant) and a sufficient amount of the recombinant polypeptide according to claim 1, a fusion protein comprising the mutated HPV-16 E7 polypeptide sequence, linked to a tat peptide, preferably the HW-I tat peptide, a polynucleotide encoding the recombinant polyeptide according to claim 1 or the fusion protein comprising the mutated HPV-16 E7 polpegptide sequence according, linked to a tat peptide, preferably the HIV-1 tat peptide, a vector comprising the polynucleotide, the polypeptide or the fusion, a cell transfected by or comprising the vector and/or an inhibitor directed against the polypeptide, the fusion protein or the polynucleotide.

15. The pharmaceutical composition according to the claim 14, which further comprises a suitable adjuvant.

16. The pharmaceutical composition according to the claim 15, wherein said adjuvant is the QuilA.

17. The pharmaceutical composition according to claim 14, which is a preventive or curative vaccine against cancer induced by a human Papillomavirus (HPV), especially the strain HPV-16.

18. The pharmaceutical composition according to the claim 17, wherein said vaccine is able to induce, when administered to a patient, a specific human humoral and/or cellular response against a human Papillomavirus, especially the strain HPV-16.

19. Use of the pharmaceutical composition according to claim 14 for the manufacture of a medicament for the treatment and/or the prevention of cancer, preferably cancer induced by a human Papillomavirus, especially the strain HPV-16.

20. A method of treatment or prevention of cancer affecting or supposed to affect a mammal (including a human), especially cancer induced by a human Papillomavirus, especially induced by HPV-16, said method comprising the steps of administrating to said mammal a sufficient amount of the pharmaceutical composition according to claim 14 in order to prevent or cure the symptoms of cancer or stop the development of tumors, in particular tumors induced by a human Papillomavirus, especially induced by HPV-16.

21. A method for obtaining a recombinant HPV E7 polypeptide from a yeast cell, which comprises the steps of:
transforming a yeast cell culture, preferably a Pichia pastoris yeast cell culture with the vector according to the claim 7,
selecting the cell(s) transformed by said vector,
culturing said selected transformed cell(s) in an adequate culture medium,
obtaining and possibly purifying a recombinant HPV-16 E7 protein from the supernatant of the culture medium of said cell(s).

22. The method according to the claim 21, wherein the culturing of the selected transformed cell(s) is made in a first step in a medium having a pH lower than 5 and a temperature comprised between about 27 and about 33° C., preferably between about 29 and about 31° C.

23. The method according to the claim 22, wherein the vector comprises a inducible promoter and wherein the culturing of the selected transformed cell(s) comprises a second step of the addition to the culture medium of a compound which induces the activity of the promoter present in the vector.

24. The method according to the claim 23, wherein the inducible promoter present in the vector is the promoter AOX1 and wherein the added compound is glycerol.

25. The method according to claim 23, wherein the culturing of the said transformed cell(s) comprises a third step wherein the pH of the medium is increased to a value of a pH higher than 5, preferably between about 5 and about 6 and wherein the temperature is maintained between about 23° C. and about 27° C. for between about 20 and about 40 hours, preferably between about 25 and about 35 hours.
